(19) United States
(12) Patent Application Publication
(10) Pub. No.: US 2014/0201865 A1
(43) Pub. Date: Jul. 17, 2014

(54) Methods for Expanding Color Palette in Dendrobium Orchids

(71) Applicant: University of Dubuque, Dubuque, IA (US)

(72) Inventor: Rasika G. Mudalige-Jayawickrama, Dubuque, IA (US)

(21) Appl. No.: 13/946,948

(22) Filed: Jul. 19, 2013

Related U.S. Application Data
(60) Provisional application No. 61/674,287, filed on Jul. 20, 2012.

(51) Int. Cl. C12N 15/82

(52) U.S. Cl. CPC C12N 15/825
USPC 800/282; 435/320.1; 800/323; 536/23.6; 530/370

(57) ABSTRACT

A nucleotide sequence encoding Flavonoid 3′-hydroxylase (F3′H) of Dendrobium, a method of producing a transgenic flower color-changed Dendrobium plant, and a transgenic flower color-changed Dendrobium plant are provided by this invention.

This application claims priority to U.S. Provisional Patent Application No. 61/674,287 filed Jul. 20, 2012, the entire contents of each of which are incorporated herein by reference.

The sequence listing submitted herewith is incorporated by reference in its entirety.

### BACKGROUND OF THE INVENTION

1. Field of the Invention

The invention disclosed herein relates generally to the fields of recombinant DNA technology directed to producing through genetic modification of anthocyanin biochemistry Dendrobium orchids having orange (pelargonidin accumulating) and blue (delphinidin-accumulating) flowers. Particularly, the invention provides methods for modifying anthocyanin biosynthesis in Dendrobium orchids through gene suppression.

2. Description of Related Art

Dendrobium, a member of the Orchidaceae is one of the largest living genera with approximately 1400 species and many man-made hybrids. Classical breeding techniques have given rise to many commercially successful hybrids with attractive flower colors and forms, long vase life, fragrance, seasonality and desirable spray length.

However, most commercial Dendrobium hybrids display predominantly purple, lavender or pink flower colors due to cyanidin and peonidin accumulation. A chemical survey of commercial Dendrobium hybrids has shown that some colors such as orange-red and blue are missing from Dendrobium flower color spectrum (Kuehnle et al., 1997, Euphytica 95: 187-194; incorporated herein in its entirety).

Unlike moth orchids and cymbidium, where the lack of a blue flower color is likely due to weak expression of flavonoid 3′,5′-hydroxylase (F3′5′H) (as described in US Patent Application Publication No. 20110191907, incorporated herein in its entirety), the limited color range within Dendrobium species can be due to the absence, mutation or over-activity of an anthocyanin biosynthetic gene. (Johnson et al., (1999) “Cymbidium hybrid dihydroflavonol 4-reductase does not efficiently reduce dihydrokaepferol to produce orange pelargonidin-type anthocyanins.” Plant J. 19:81-85; incorporated herein in its entirety).

Although substrate specificity of dihydroflavonol 4-reductase (DFR) may explain the absence of certain colors among some ornamental plants, Obsuwan et al. has shown that Dendrobium DFR can efficiently catalyze reduction of dihydrokaempferol (DHK), dihydroquercetin (DHQ), and dihydromyricetin (DHM) resulting in the production of pelargonidin, cyanidin and delphinidin with no substrate specificity. (Obsuwan et al., (2007) “Functional characterization of dendrobium and oncidium dfr in petunia hybrida model.” Acta Hort. (ISHS) 764:137-144; incorporated herein in its entirety).

DFR substrate specificity in orchids has been previously investigated. For example, DFR from Petunia and Cymbidium (an orchid) cannot reduce DHK efficiently, explaining the lack of pelargonidin-accumulating orange flowers even in the absence of competing enzymes flavonoid 3′-hydroxylase (F3′H) and F3′5′H (Forkmann and Ruhnau, 1987, “Distinct substrate specificity of dihydroflavonol 4-reductase from flowers of Petunia hybrida.” Z. Naturforsch. 42c: 1146-1148; Gerats et al., 1982, “Genetic control of the conversion of dihydroflavonols into flavanols and anthocyanins in flowers of Petunia hybrid,” Planta 155: 364-68; Johnson et al., (1999) “Cymbidium hybrid dihydroflavonol 4-reductase does not efficiently reduce dihydrokaepferol to produce orange pelargonidin-type anthocyanins”, Plant J. 19:81-85; each of which is incorporated herein in its entirety.).

Johnson et al. (2001, “Regulation of DNA binding and trans-activation by a xenobiotic stress-activated plant transcription factor” J. Biol. Chem. 276:172-178; incorporated herein in its entirety) has demonstrated that substrate specificity is found in DFR from Cymbidium orchid by heterologous expression in a Petunia host. Substrate specificity was not, however, found in Dendrobium DFR inside a similar Petunia host (Mudalige-Jayawickrama et al., 2005, “Cloning and characterization of two anthocyanin biosynthetic genes from Dendrobium orchid. J. Amer. Soc. Hort. Sci. 130:611-618; Obsuwan et al., 2007, Id.; each of which is incorporated herein in its entirety). Therefore, the rarity of pelargonidin-accumulating flowers in Dendrobium may be due to the competition from a robust F3′H enzyme that siphons off a necessary intermediate dihydrokaempferol (DHK) into purple pathway. (Mudalige-Jayawickrama et al., (2005), Id.).

Thus, there is a need in the art to delineate the biochemical basis of Dendrobium flower color by isolating and characterizating anthocyanin biosynthetic genes, and particular the gene encoding F3′H, in order to determine the reason(s) for lack of blue delphinidin and rarity of orange pelargonidin among commercial Dendrobium hybrids.

### SUMMARY OF THE INVENTION

It is against the above background that the present invention provides certain advantages and advancements over the prior art.

Although this invention is not limited to specific advantages or functionality, it is noted that the invention provides isolated nucleic acid molecules having a nucleotide sequence encoding a polypeptide having an amino acid sequence as set forth in SEQ ID NO: 2 or an amino acid sequence having at least 90% homology to the amino acid sequence as set forth in SEQ ID NO: 2.

In some embodiments, the isolated nucleic acid molecule comprises a nucleotide sequence as set forth in SEQ ID NO: 1.

In some embodiments, the polypeptide may be Flavonoid 3′-hydroxylase (F3′H) from Dendrobium.

In another aspect, the invention provides recombinant genetic constructs, comprising the nucleic acid molecule as set forth in SEQ ID NO: 1 and a suppressor of the nucleic acid molecule as set forth in SEQ ID NO: 1, wherein the suppressor may be a sense or an antisense suppressor.

In another aspect, the invention provides methods for producing a transgenic plant, comprising transfecting a plant with a gene construct comprising the nucleic acid molecule as set forth in SEQ ID NO: 1 and a suppressor of the nucleic acid molecule as set forth in SEQ ID NO: 1, wherein the suppressor may be a sense or an antisense suppressor and wherein the genetic construct is expressed in transgenic plant cells.

In some embodiments, the transgenic plant is a flower color-changed plant and wherein the plant is a native-color plant.

In further embodiments, the flower color-changed plant and the native-color plant are members of the Orchidaceae family plant. In yet further embodiments, the flower color-changed plant and the native-color plant are Dendrobium orchids.

In another aspect, the invention provides methods for producing a flower color-changed plant having an orange flower, which comprises transfecting a native-color plant having a purple flower with the gene construct comprising the nucleic acid molecule as set forth in SEQ ID NO: 1 and a suppressor of the nucleic acid molecule as set forth in SEQ ID NO: 1, wherein the suppressor may be a sense or an antisense suppressor and wherein the genetic construct is expressed in transgenic plant cells.

In some embodiments of the method for producing a flower color-changed the flower color-changed plant and the native-color plant are members of the Orchidaceae family plant. In further embodiments, the flower color-changed plant and the native-color plant are Dendrobium orchids.

In another aspect, the invention provides a flower color-changed plant produced by transfecting a plant with the gene construct comprising the nucleic acid molecule as set forth in SEQ ID NO: 1 and a suppressor of the nucleic acid molecule as set forth in SEQ ID NO: 1, wherein the suppressor may be a sense or an antisense suppressor and wherein the genetic construct is expressed in transgenic plant cells.

In some embodiments, the flower color-changed plant is an Orchidaceae family plant. In further embodiments, the flower color-changed plant is a Dendrobium orchid.

In another aspect, the invention provides a flower color-changed plant produced by transfecting a native-color plant having a purple flower with the gene construct comprising the nucleic acid molecule as set forth in SEQ ID NO: 1 and a suppressor of the nucleic acid molecule as set forth in SEQ ID NO: 1, wherein the suppressor may be a sense or an antisense suppressor and wherein the genetic construct is expressed in transgenic plant cells.

In some embodiments, the flower color-changed plant is an Orchidaceae family plant. In further embodiments, the flower color-changed plant according to claim 14, which is a Dendrobium orchid.

In another aspect, the invention provides a flower color-changed plant having the gene construct comprising the nucleic acid molecule as set forth in SEQ ID NO: 1 and a suppressor of the nucleic acid molecule as set forth in SEQ ID NO: 1, wherein the suppressor may be a sense or an antisense suppressor. In some embodiments, the flower color-changed plant is an Orchidaceae family plant. In further embodiments, the flower color-changed plant is a Dendrobium orchid.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows that Flavonoids are synthesized via a complex biochemical pathway known as the phenylpropanoid pathway. (A) Typical purple Dendrobium hybrid. (B) Rare pelargonidin accumulating mutant. (C) Anthocyanin biosynthetic pathway with the enzyme abbreviations. Dihydrokaempfeol (DHK) intermediate is surrounded by the red circle.

FIG. 2 shows chemical analysis of the purple Dendrobium flower UHSO3 and the Petunia W80 mutant flowers transformed with 35S:Antirrhinum Dfr and 35S:Dendrobium Dfr. The pelargonidin and orange color in Den-Dfr transformant. (Obsuwan et al., 2007, Id.).

FIG. 3 shows Dendrobium F3′H sequence analysis. (A) Multiple sequence alignments of deduced amino acid sequences of Dendrobium-F3′H and other plant species using CLUSTALW program. The “*” represent conserved amino acids; “:” represent similar amino acids substitutions. Dendrobium_Jaquelyn_Thomas (SEQ ID NO.: 12); Lilium_hybrid (SEQ ID NO.: 13); Sorghum_bicolor (SEQ ID NO.: 14); Zea_{—}mays (SEQ ID NO.: 15); Allium_{—}cepa (SEQ ID NO.: 16); Antirrhinum_{—}majus (SEQ ID NO.: 17); Torenia_hybrid (SEQ ID NO.: 18); Malus_{—}x_{—}domestica (SEQ ID NO.: 19); Matthiola_{—}incana (SEQ ID NO.: 20); Pelargonium_{—}x_{—}hortorum (SEQ ID NO.: 21). (B) Phylogenetic relationships determined by amino acid sequence similarity (PHYLIP version 3.5c).

FIG. 4 shows photographs of agarose gel electrophopretic analyses of RT-PCR products of F3′H and DFR in different floral organs of D. Jaquelyn Thomas ‘Uniwai Prince’ (UHSO3) and D. Icy Pink ‘Sakura’ (K1224) orchids. Different stages of floral buds used for analysis are shown on top. F3′H mRNA is absent in Pelargonidin-accumulating flower buds (K1224). Actin was used to normalize RNA loading levels.

FIG. 5 shows a schematic representation of different strategies that are being used to increase the color pallete of commercial Dendrobium hybrids. Shutting down the F3′H enzyme is an essential part of a successful strategy.

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Methods well known to those skilled in the art can be used to construct genetic expression constructs and recombinant cells according to this invention. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

Further, to generate transgenic plants a Particle Inflow Gun may be used to deliver gold and/or tungsten particles carrying the gene construct. (Finer et al., (1992) “Development of the particle inflow gun for DNA delivery to plant cells.” Plant Cell Reports 11:232-238; Vain et al., (1993) “Development of the Particle Inflow Gun.” Plant Cell Tiss Org Cult 33:237-246).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or can not be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

Anthocyanidins are water-soluble pigments (colored flavonoid glycosides) that accumulate in plant cell vacuoles giving characteristic colors to flowers and fruits and can be responsible for red-pink cyanidin, orange pelargonidin, and blue delphinidin in flowers. Production of the three primary classes of anthocyanidins by the phenyl propanoid pathway is controlled by the availability of the colorless substrates dihydrokaempferol (DHK), dihydroquercetin (DHQ), and dihydromyricetin (DHM) and the activities of flavonoid 3′-hydroxylase (F3′H), flavonoid 3′,5′-hydroxylase (F3′5′H), and Dihydroflavonol 4-reductase (DFR). Conversion of those three dihydroflavonoids into leucoanthocyanidins is a required step in anthocyanin biosynthesis and is catalyzed by DFR.

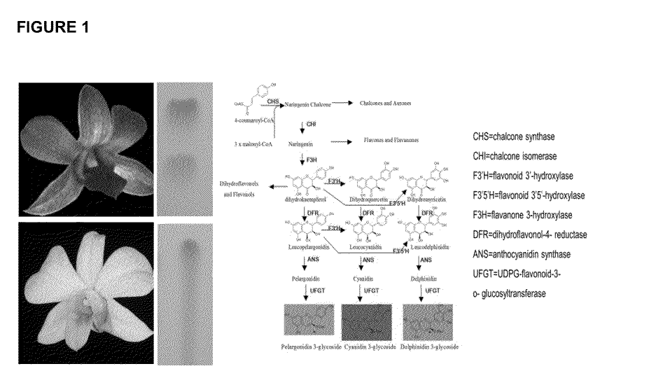

Dendrobium, the largest genus of the orchid family, display predominantly, purple, lavender and pink flowers due to cyanidin and peonidin accumulation (FIG. 1A). Blue delphinidin is absent in Dendrobium hybrids while orange pelargonidin (FIG. 1B) is found in a few rare mutants (FIG. 1; Kuehnle et al., 1997, Id.).

DFR is important in flower color due to its substrate specificity. Substrate specificity of DFR explains the absence of certain colors among some ornamental plants, which make this enzyme an important target for flower color manipulation through genetic engineering. In order to characterize DFR in two major subtropical orchids, full-length cDNA clones encoding DFR are isolated using a RT-PCR based technique from petals of hybrid plants resulting from Dendrobium×Icy Pink ‘Sakura’ and Oncidium×Gower Ramsey genetic crosses.

The substrate specificity of Dendrobium DFR and Oncidium DFR were investigated by genetic transformation of the mutant Petunia line W80 that predominantly accumulates DHK. Chemical analysis of transformed lines revealed that both Dendrobium DFR and Oncidium DFR can efficiently catalyze the reduction of DHK, DHQ and DHM and can result in the production of pelargonidin, cyanidin and delphinidin with no substrate specificity.

In order to understand the reason for lack of blue delphinidin and rarity of orange pelargonidin among commercial Dendrobium hybrids, the biochemical basis of Dendrobium flower color was delineated as set forth herein by isolation and characterization of certain anthocyanin biosynthetic genes. As a consequence, disclosed herein are methods for expanding the available flower colors for Dendrobium and other orchids through genetic manipulation.

In orchids, flavonoids are synthesized via a complex biochemical pathway known as the phenylpropanoid pathway (FIG. 1). The first committed step of flavonoid biosynthesis is condensation of 3 molecules of malonyl-CoA with a single molecule of 4-coumaroyl-CoA to form chalcone, catalyzed by the enzyme chalcone synthase (CHS). Chalcone is then isomerized to naringenin, a colorless flavonone, by chalcone isomerase (CHI). Naringenin is subsequently hydroxylated by flavanone 3-hydroxylase (F3H) to form dihydrokaempferol (DHK), a common intermediate to several flavonoid species. DHK can be hydroxylated at the 3′ position of the B ring to form dihydroquercetin (DHQ) or at both the 3′ and 5′ positions to form dihydromyricetin (DHM); the DHQ reaction is catalyzed by Flavonoid 3′-hydroxylase (F3′H) and the DHM reaction is catalyzed by Flavonoid 3′,5′-hydroxylase (F3′5′H). DHK is an intermediate that can be utilized by all three branches of the pathway to produce orange pelargonidin, purple cyanidin or blue delphinidin as the final anthocyanidin. Dihydroflavonol 4-reductase can accept DHK, DHQ or DHM to produce orange, purple and blue colors, respectively.

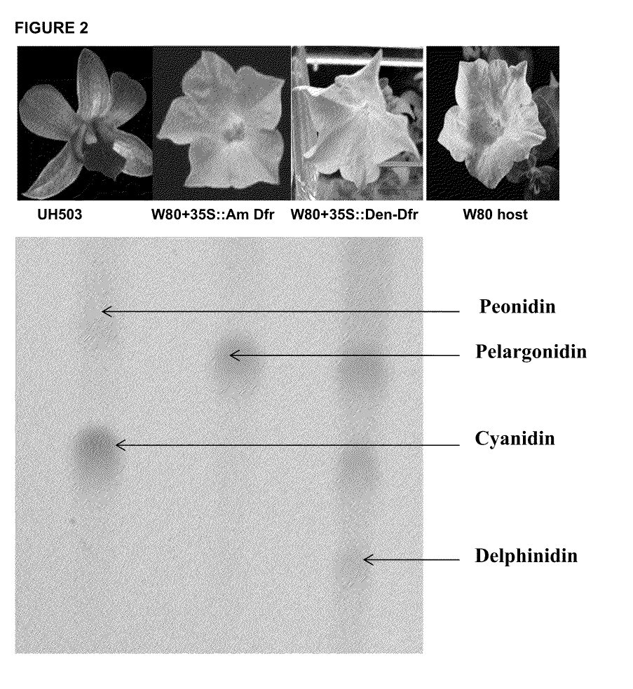

Substrate specificity of DFR was investigated through heterologous expression of Dendrobium DFR in a petunia host. Petunia DFR cannot efficiently reduce DHK to produce orange pelargonidin-accumulating flowers even in the absence of competing enzymes flavonoid 3′-hydroxylase (F3′H) and (flavonoid 3′,5′-hydroxylase) F3′5′H (FIG. 2 W80). Zea mays DFR enzyme efficiently catalyzed the reduction of DHK to produce novel transgenic orange colored Petunia (Meyer et al. 1987, “A new petunia flower colour generated by transformation of a mutant with a maize gene.” Nature 330: 677-678). However, Orchid DFR enzymes produced contradicting results when inserted into the same petunia host. The Cymbidium orchid DFR did not reduce DHK to make pelargonidin efficiently (Johnson et al., 1999) whereas Dendrobium DFR was able to make orange pelargonidin (FIG. 2; Obsuwan et al., 2007)

Surprisingly and unexpectedly, disclosed herein is the finding that Dendrobium DFR is capable of accepting the precursors of all three colors, orange, purple and blue in petunia. Therefore, substrate specificity of DFR does not determine the flower color of Dendrobium and is not the reason for predominance of purple color in Dendrobium hybrids. Thus, it became apparent that enzyme competition among DFR, F3′H, and F3′5′H can determine flower color of Dendrobium orchid.

The predominance of cyanidin can occur either due to substrate specificity of the DFR enzyme or enzymatic competition among DFR, flavonoid 3′ hydroxylase (F3′H) and flavonoid 3′5′ hydroxylase (F3′5′H) for the common substrate dihydrokaempferol.

An explanation for the observed color patterns in orchids is that rare pelargonidin flowers must be deficient in F3′H, eliminating enzyme competition for DHK so that DHK is catalyzed directly by DFR towards pelargonidin.

Accordingly, in one aspect, the invention provides a gene (SEQ ID NO: 1) encoding F3′H from Dendrobium (SEQ ID NO: 2). Deduced amino acid sequence of the full gene is 69-70% similar to F3′H sequences from other orchid species. F3′H is expressed in all bud stages with the highest expression in mature buds. Expression declines as the flower opens. F3′H is mutated in the orange, pelargonidin-accumulating mutant, suggesting lack of competition from F3′H may lead to novel orange pelargonidin accumulators.

Discovery of Dendrobium F3′H gene permits F3′H gene expression to be evaluated and for it to be determined that rare pelargonidin flowers do not show F3′H expression. Moreover, reduction in F3′H activity via gene suppression can be used to produce orange Dendrobium hybrids and breeding materials.

Previous results on a different orchid, Cymbidium, have shown that the predominance of purple anthocyanidins, cyanidin and peonidin, is due to substrate specificity of Dihydrofalavonol 4-reductase enzyme (Johnson et al., 1999, Id.) However, as shown herein substrate specificity is not the biochemical basis for the color patterns shown in naturally occurring Dendrobium orchids.

First, amino acid residues that render substrate specificity to other DFR enzymes, e.g., Petunia, are not shared by the Dendrobium DFR. Second, heterologous expression of Dendrobium DFR in a petunia mutant resulted in the production of orange pelargonidin in the transgenic line. Therefore, the purple predominance in Dendrobium orchids is surprisingly and unexpectedly due to the competition among DFR, F3′H and F3′5′H to accept the common intermediate dihydrokaempferol (DHK).

Unlike predominantly purple Dendrobium orchids, as disclosed herein rare orange pelargonidin-accumulating mutants surprisingly and unexpectedly accept DHK due to the absence of strong competition from the F3′H enzyme similar to a pelargonidin accumulating mutant, Dendrobium Icy Pink “Sakura”, that does not express F3′H.

In preferred embodiments, the invention provides methods for rerouting the anthocyanin biosynthetic pathway from purple cyanidin towards orange pelargonidin by inhibiting F3′H enzyme activity in a purple Dendrobium orchid. In certain embodiments, genetic suppression is accomplished by RNA interference mediated by introduction of siRNA into plant cells. This method preferably does not produce chimeras of transformed and non-transformed sections in a single plant because gene silencing occurs through an RNA interference pathway, which allows gene suppression to occur in a systemic manner.

### EXAMPLES

The Examples that follow are illustrative of specific embodiments of the invention, and various uses thereof. They are set forth for explanatory purposes only, and are not to be taken as limiting the invention.

### Example 1

### Isolation of Dendrobium Flavonoid 3′-Hydroxylase

Inflorescences of Dendrobium Jaquelyn Thomas ‘Uniwai Prince’ (UH 503) were harvested from University of Dubuque greenhouse grown plants. Total RNA was extracted from unopened buds according to the method of Champagne and Kuehnle (2000), “An effective method for isolating RNA from tissues of Dendrobium.” Lindleyana 15:165-168, which is incorporated by reference in its entirety.

cDNA was synthesized from 5 micrograms of total RNA using 200 units of SuperScript III reverse transcriptase (Invitrogen, Carlsbad, Calif.) according to conventional methods. Oligo dT (dT16 or dT20-T7) primers were used for first strand cDNA synthesis. The reaction was stopped by incubation of the mixture at 70° C. for 15 min. The RNA template was removed by incubating the reaction mixture with 2 units of RNase H (Promega, Madison, Wis.) at 37° C. for 20 minutes. Resultant cDNA strands were used as the template for RT-PCR with degenerate primers targeted to the specific conserved regions of F3′H amino acid sequence alignment of publicly available monocot and some dicot sequences. (Arabidopsis thaliana:AF271651, Oryza sativa:AC021892, Pelargonium x hortorum:AF315465, Petunia hybrida:AF155332, Torenia hybrida:AB0057673, and Sorghum bibolor:AY675075, and Zea mays: HQ699781).

Two degenerate primers, Den-degen-F3′H-for GGNGTNGAYGTNAARGG (SEQ ID NO: 3) and Den-F3′H-Rev CCRTANGCYTCYTCCAT (SEQ ID NO: 4), were used at a 1.20 micromolar final concentration in a 25 microliter PCR reaction. Initial denaturation was done at 95° C. for 2 minutes followed by 30 cycles of amplification at 94° C. for 30 seconds, 49° C. for 30 seconds and 68° C. for 30 seconds. A final extension was carried out at 68° C. for 7 minutes. The resultant products were separated on a 1.5% agarose gel in 1XTAE electrophoresis buffer. A gel fragment containing a 180 base pair band was excised and cleaned using Qiagen MinElute Gel extraction kit, and was cloned into a pGEM-T easy vector system according to conventional methods and the supplier's instructions.

A partial sequence of the putative Dendrobium F3′H was determined by sequencing cloned cDNA with T7 and Sp6 primers. The remainder of the F3′H gene was isolated using 5′ and 3′ RACE (Rapid Amplification of cDNA ends). 3′RACE was performed using this same cDNA with a gene-specific forward primer ATGACGGCGACGTTGATTCATG (SEQ ID NO: 5) and T7 primer TAATACGACTCACTATAGGG (SEQ ID NO: 6) at a 10:1 concentration ratio. Amplification for 35 cycles was performed under amplification conditions comprising 94° C. for 30 seconds, 55° C. for 30 seconds, and 68° C. for 30 seconds followed by a final extention at 68° C. for 7 minutes. Resultant PCR products were gel purified, cloned into pGEM-T easy vector and sequenced as described above.

For 5′RACE this same RNA was used with a 5′RACE kit from Invitogen. Three primers were designed from the isolated partial clone sequence. Den-F3′H-end primer, TTAAACATCTTTAGGATATGC (SEQ ID NO: 7) was used as the gene specific primer to synthesize the first strand using SuperScript III reverse transcriptase enzyme. Primary PCR was performed for 30 cycles using Den-F3′H-12 primer GAGCCCATAAGCCTCTTCCAT (SEQ ID NO: 8) at 94° C. for 30 seconds, 55° C. for 30 seconds and 68° C. for 1.40 minutes. Primary PCR product was diluted 1:10 in sterile water. Diluted primary PCR product was used as the template to carry out secondary PCR. Nested PCR was carried out with primer Den-F3′H-11 GATTCTTCGCCCAGCGCCGAACGG (SEQ ID NO: 9) at 94° C. for 30 sec, 55° C. for 30 sec, and 68° C. for 1.30 minutes. Resultant PCR product was gel purified and inserted into a pGEM-T easy vector system as described above. Amplified DNA comprising full length F3′H-encoding sequence was cloned according to the 5′ and 3′ RACE sequences by PCR amplification with the Den-F3′H-start ATGGGCTTCATTTTCCTCTTTG (SEQ ID NO: 10) and Den F3′H-end TTAAACATCTTTAGGATATGC (SEQ ID NO: 11) primers.

PCR amplification for 30 cycles was carried out at 94° C. for 30 seconds, 55° C. for 30 seconds, and 68° C. for 1.40 minutes. Resultant PCR product comprising a F3′H-encoding complete open reading frame was cloned into pGEM-T easy vector for further manipulations.

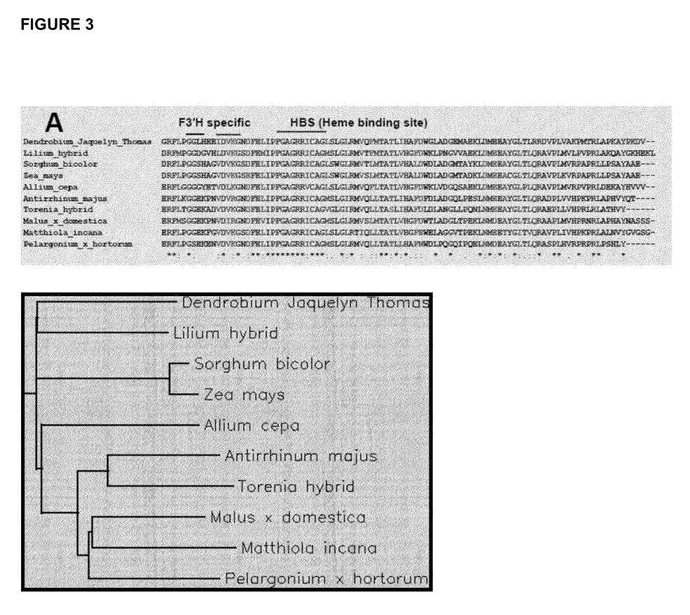

Dendrobium F3′H from Dendrobium orchid is 77% similar and 66% identical to the closest F3′H sequence found in GenBank (FIG. 3). Signature sequences that are specific to F3′H are conserved in DenF3′H. Amino acid sequence analysis suggests that it is most closely related to Lilioid monocots, followed by other grass monocots.

### Example 2

### Expression Profiles in Dendrobium

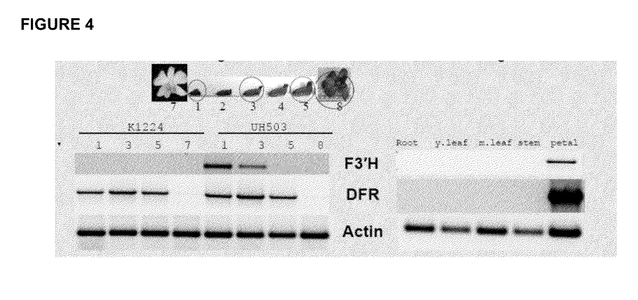

Temporal expression profile for F3′H from Dendrobium was determined for different stages of flower buds and spatial expression profile was determined for different plant organs. Thin layer chromatography of petals was performed according to the method of Kuehnle et al. (1997), Id. and Irani and Grotewald (2005, “Light-induced morphological alteration in anthocyanin-accumulating vacuoles of maize cells,” BMC Plant Biol. 5: 7, which is incorporated in its entirety). The results are shown in FIG. 4.

RT-PCR were performed using total RNA extracted from different plant organs (structures) to determine spatial expression profile while temporal expression profile of F3′H was assessed using RNA extracted from different floral bud stages.

As shown previously, heterologous expression of Dendrobium-Dfr in a mutant petunia host indicated that the Dendrobium-DFR is capable of accepting DHK as a substrate to produce orange pelargonidin.

Qualitative expression analyses of F3′H by RT-PCR demonstrates that pelargonidin-accumulating mutants such as K1224 does not express F3′H. Therefore, the absence of competing enzyme, F3′H, appear to be a prerequisite to convert DHK to orange pelargonidin via the activity of DFR in Dendrobium orchids.

### Example 3

### Transfection Procedures

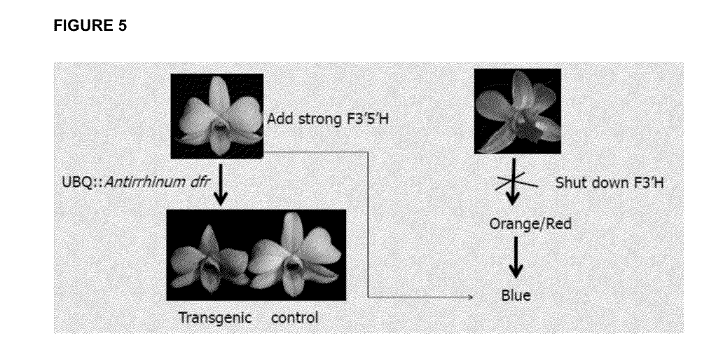

Dendrobium flower color can be modified through suppression of F3′H enzyme activity using sense and antisense suppression strategies (FIG. 5). To generate transgenic plants a Particle Inflow Gun can be used to deliver gold and/or tungsten particles carrying a recombinant genetic construct as set forth herein. (Finer et al., (1992) “Development of the particle inflow gun for DNA delivery to plant cells.” Plant Cell Reports 11:232-238; Vain et al., (1993) “Development of the Particle Inflow Gun.” Plant Cell Tiss Org Cult 33:237-246).

Briefly, in one example, cell transformation procedure using the Particle Inflow Gun can be carried out as follows:

(a) Sterilization of particles.

1. Suspend 50 mg of either tungsten or gold particles in 500 μL of 95% ethanol (prepared from 100% ethanol) and let set for 15 minutes. 2. Spin gently to pellet the particles and remove the supernatant. Wash with 500 μL sterile dH2O 3×. 3. Resuspend the pellet in 330 μL sterile dH2O to a final concentration 0.15 mg/μL. The actual volume is not critical, you simply want to a concentrated stock of sterile particles. This volume worked well for me with my plasmid preps.

(b) Precipitation of DNA upon the particles.

1. Precipitate 5--15 μg of DNA construct (as described above) upon 2.25 mg of 0.7-μm diameter tungsten (M10, 0.7-μm diameter on average; Sigma) or 1-μm diameter gold particles (Bio-Rad Laboratories). 2. First, remove the appropriate amount of sterilized particles (15 μL in my case) and place in a sterile eppendorf tube. The next few steps are then completed as quickly as possible. 3. Add the appropriate DNA(s) in a total volume of 15 μL. Mix well. For control experiments, dH2O is substituted for the

DNA solution. For cotransformation experiments an additional 10--15 μg of a second plasmid DNA are added as appropriate. 4. Then add 25 μL of 2.5 M CaCl2, mix well. 5. This is followed by 10 μL of 100 mM spermidine (prepared fresh from 1M stock), and mixed well. 6. After the addition of spermidine, the solution is incubated on ice for 5 min during which time the particles settled. These can set for at least 1 hour with no noticeable effect upon transformation efficiency. 7. The top 45 μL are carefully removed and a 10-ul aliquot of the pellet is removed and placed on top of the filter mesh of either a 13-mm Swinney (Gelman Laboratory, Ann Arbor Mich.) or Swinnex (Millipore, Billerica Mass.) filter. The filter was screwed into a Leur-lock attachment connected to the centered collar (see bombardment procedure below).

(c) Preparation of cells.

1. Filter cells through two layers of cheesecloth to remove bacterial mats. 2. Cells are collected by centrifugation (2 min at ˜600×g) and re-suspended at a cell density of ˜0.5--1×104 cells/ml) in Buffer C (85% (v/v) 10 mM KOH, 5 mM KCI, 5 mM HEPES adjusted to pH 7.0 with HCl, and 15% ABW). 3. A 1-ml aliquot of the cell suspension is placed into a 35-mm sterile Petri dish and swirled to achieve an even, thin layer across the bottom of the dish.

(d) Bombardment procedure.

1. The top 45 μL of the precipitation mixture (see above) are carefully removed and a 10-μl aliquot of the pellet is placed on top of the filter mesh of either a 13-mm Swinney (Gelman Laboratory, Ann Arbor Mich.) or Swinnex (Millipore, Billerica Mass.) filter. 2. The filter is screwed into a Leur-lock attachment connected to the centered collar. 3. The Petri dish top from the cell preparation above is removed and the bottom placed upon the stand. 4. The plexiglass door is attached, screwed tight, and a vacuum pulled to between 25--30 mm Hg. 5. A 50-ms burst of pressurized helium gas is released into the chamber through the filter unit by the action of the timer relay-driven solenoid (there will be a splash). 6. The vacuum is gently broken and the cell suspension is diluted in 6 ml of ABW media. 7. Cells are grown for three days without selection at 28° C. in a humidity chamber, which is a sealed plastic-ware container with damp paper towels lining the bottom. 8. Over the next three days the culture is expanded to 10 ml by the daily addition of 1 mL of ABW. 9. After three days, the cells are counted and freshly prepared paromomycin added to a final concentration of 20--50 μg/ml (determined empirically). Generally 20 μg/mL works well for small populations of cells and 50 μg/mL works better for selection in mass. 10. Cells are grown for 2 days at 28 ° C. before assessment of transformation efficiency.

### Example 4

### Production of Transformed Orchids

Petunia leaf discs were transformed with Dfr constructs using Agrobacterium mediated transformation (Obsuwan et al. 2007, Id.). Dendrobium Icy Pink ‘Sakura’ PLBs were transformed with UBQ3::Antirrhinum Dfr via Biolistic bombardment (BIO-RAD).

### REFERENCES

Champagne, M. M. and A. R. Kuehnle. 2000. An effective method for isolating RNA from tissues of Dendrobium. Lindleyana 15:165-168.

Felsensein J. 1993. PHYLIP (Phylogeny Inference Package) version 3.5c, Department of Genetics, University of Washington

Johnson E. T., Yi H., Shin B., Oh B. J., Cheong H., and G. Choi. 1999. Cymbidium hybrid dihydroflavonol 4-reductase does not efficiently reduce dihydrokaepferol to produce orange pelargonidin-type anthocyanins. Plant J. 19:81-85.

Kuehnle, A. R., D. H. Lewis, K. R. Markham, K. A. Mitchell, K. M. Davies, and B. R. Jordan. 1997. Floral flavonoids and pH in Dendrobium orchid species and hybrids. Euphytica 95:187-194.

Mudalige-Jayawickrama R. G., Champagne M. M., Hieber A. D. and A. R. Kuehnle 2005. Cloning and characterization of two anthocyanin biosynthetic genes from Dendrobium hybrid. J. Amer. Soc. Hort. Sci. 130(4):611-618.

Obsuwan, K., Hieber, D. A., Mudalige-Jayawickrama, R. G. and A. R. Kuehnle. 2007. Functional characterization of dendrobium and oncidium dfr in petunia hybrida model. Acta Hort. (ISHS) 764:137-144

Thompson J. D., Higgins D. G. and Gibson T. J. 1994. Improving the sensitivity of progressive multiple sequence alignment through sequence alignment through sequence weighting, position-specific gap penalties and weight matrix choice. Nucleic Acids Res. 22: 4673-4680.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. An isolated nucleic acid molecule having a nucleotide sequence encoding a polypeptide having an amino acid sequence as set forth in SEQ ID NO: 2 or an amino acid sequence having at least 90% homology to the amino acid sequence as set forth in SEQ ID NO: 2.

2. The nucleic acid molecule of claim 1, wherein the nucleotide sequence is SEQ ID NO: 1.

3. The polypeptide of claim 1, wherein the polypeptide is a Flavonoid 3′-hydroxylase (F3′H) of Dendrobium.

4. A recombinant genetic construct, comprising the nucleic acid molecule of claim 1 and a suppressor of the nucleic acid molecule of claim 1, wherein the suppressor is a sense or an antisense suppressor.

5. A method for producing a transgenic plant, comprising transfecting a plant with the gene construct as defined in claim 4 and expressing the recombinant genetic construct in transgenic plant cells.

6. The method of claim 5, wherein the transgenic plant is a flower color-changed plant and wherein the plant is a native-color plant.

7. The method of claim 6, wherein the flower color-changed plant and the native-color plant are members of the Orchidaceae family plant.

8. The method of claim 7, wherein the flower color-changed plant and the native-color plant are Dendrobium orchid.

9. A method for producing a flower color-changed plant having an orange flower, which comprises transfecting a native-color plant having a purple flower with the gene construct as defined in claim 4 and expressing recombinant genetic construct in transgenic plant cells.

10. The method of claim 9, wherein the flower color-changed plant and the native-color plant are members of the Orchidaceae family plant.

11. The method of claim 10, wherein the flower color-changed plant and the native-color plant are Dendrobium orchid.

12. A flower color-changed plant produced by the method of claim 5.

13. The flower color-changed plant according to claim 12, which is an Orchidaceae family plant.

14. The flower color-changed plant according to claim 12, which is Dendrobium orchid.

15. A flower color-changed plant produced by the method of claim 9.

16. The flower color-changed plant according to claim 15, which is an Orchidaceae family plant.

17. The flower color-changed plant according to claim 15, which is Dendrobium orchid.

18. A flower color-changed plant comprising in cells thereof the recombinant genetic construct as defined in claim 4.

19. The flower color-changed plant according to claim 18, which is an Orchidaceae family plant.

20. The flower color-changed plant according to claim 19, which is Dendrobium orchid.


SEQUENCE LISTING (21 sequences)

SEQ ID NO: 1 - DNA, 1548 bp, Dendrobium  Jaquelyn Thomas ′Uniwai Prince′
  CDS (1)..(1548)
atgggcttcattttcctctttgtcactttcatcctcacctatgtccacctccgctccggcaatcaccggcggcggataggccgccgccttccgccgggaccgaaagattggccgatcattgggaaccttccgcaactcggccccaaacctcaccagacgctacacgccctttcaaaaaccttcggcccaatcctcagcctccgtttcggcgccgtcgatgtcgtcgtcgcctcctccgccgccgccgcttctcaatttctccgcacacacgacgcaaatttcagcggccggccgcccaactccggcgccgagcacgtcgcgtacaactaccaggatctcgtattcgcaccgtacggtgcgcggtggcgcatgctgaggcgtctatgcgccttgcatctattttccgcgaaagcgatggaagattttcggcacgtgcgggcaggcgaggtggagcggctcgtgcggcgattagcggagaaggcgggagaggcggtagacgtgggtggggaggtgaacacctgtgcgaccaatgcgctgacacgtgcgacggtggggcggcgggtgttcggggaaaaggaggagggggaaggtgcggaggagtttaaggagatggtggtggagcttatgaagctcgccggagtttttaatataggggattttgtccccggcttgggatggcttgatttacagggagtggtgaagaagatgaagaagttgcatagaagatttgatgaattcttcgatggaataattgcagagcatagagaagcagaagagaaagctgattctgatggatcaaaacgcagcgatatgctcagcatactcattgggctgaaagaggaagcttgtggagaaggaatcaagcttacagacacagacatcaaggctctcctactgaatctttttacagccggaactgacacgacgtctagcacagtggaatgggctttggccgagctgattcgacacccaaatctcctaaagcaagcgcaaatcgagctcgactccgtcgtcggatccgatcggctcgtctccgagtccgatctccccaacctccccttcctccaagccatcgtcaaagagacctttcgcctccatccctcaaccccgctctcccttccgcgcattgcttccaaggactgtgagatcgatggctacttgattcctgcaggctccactctcttggtcaatgtctggtccattgctcgagaccccatcatgtggcccgaccacccgctagcttttcaacctggacggtttcttccaggcggtctgcatgaggaaatcgacgtcaaagggaacgattttgagctcattccgttcggcgctgggcgaagaatctgtgcagggttgagtttaggtttgcgaatggttcaattcatgacggcgacgttgattcatgccttcgattggggtttggccgacggggaaatggctgagaagctcgacatggaagaggcttatgggctcacgcttcgcagagatgtgccgctagtcgctaagccgatgactcggctagcccccaaagcatatcctaaagatgtttaa

SEQ ID NO: 2 - protein, 515 aa, Dendrobium  Jaquelyn Thomas ′Uniwai Prince′
MGFIFLFVTFILTYVHLRSGNHRRRIGRRLPPGPKDWPIIGNLPQLGPKPHQTLHALSKTFGPILSLRFGAVDVVVASSAAAASQFLRTHDANFSGRPPNSGAEHVAYNYQDLVFAPYGARWRMLRRLCALHLFSAKAMEDFRHVRAGEVERLVRRLAEKAGEAVDVGGEVNTCATNALTRATVGRRVFGEKEEGEGAEEFKEMVVELMKLAGVFNIGDFVPGLGWLDLQGVVKKMKKLHRRFDEFFDGIIAEHREAEEKADSDGSKRSDMLSILIGLKEEACGEGIKLTDTDIKALLLNLFTAGTDTTSSTVEWALAELIRHPNLLKQAQIELDSVVGSDRLVSESDLPNLPFLQAIVKETFRLHPSTPLSLPRIASKDCEIDGYLIPAGSTLLVNVWSIARDPIMWPDHPLAFQPGRFLPGGLHEEIDVKGNDFELIPFGAGRRICAGLSLGLRMVQFMTATLIHAFDWGLADGEMAEKLDMEEAYGLTLRRDVPLVAKPMTRLAPKAYPKDV

SEQ ID NO: 3 - DNA, 17 bp, Artificial Sequence
  Synthetic oligonucleotide
ggngtngaygtnaargg

SEQ ID NO: 4 - DNA, 17 bp, Artificial Sequence
  Synthetic oligonucleotide
ccrtangcytcytccat

SEQ ID NO: 5 - DNA, 22 bp, Artificial Sequence
  Synthetic oligonucleotide
atgacggcgacgttgattcatg

SEQ ID NO: 6 - DNA, 20 bp, Artificial Sequence
  Synthetic oligonucleotide
taatacgactcactataggg

SEQ ID NO: 7 - DNA, 21 bp, Artificial Sequence
  Synthetic oligonucleotide
ttaaacatctttaggatatgc

SEQ ID NO: 8 - DNA, 21 bp, Artificial Sequence
  Synthetic oligonucleotide
gagcccataagcctcttccat

SEQ ID NO: 9 - DNA, 24 bp, Artificial Sequence
  Synthetic oligonucleotide
gattcttcgcccagcgccgaacgg

SEQ ID NO: 10 - DNA, 22 bp, Artificial Sequence
  Synthetic oligonucleotide
atgggcttcattttcctctttg

SEQ ID NO: 11 - DNA, 21 bp, Artificial Sequence
  Synthetic oligonucleotide
ttaaacatctttaggatatgc

SEQ ID NO: 12 - protein, 98 aa, Dendrobium  Jaquelyn Thomas
GRFLPGGLHEEIDVKGNDFELIPFGAGRRICAGLSLGLRMVQFMTATLIHAFDWGLADGEMAEKLDMEEAYGLTLRRDVPLVAKPMTRLAPKAYPKDV

SEQ ID NO: 13 - protein, 100 aa, Lilium hybrid
DRFMPGGDGVHLDVKGSDFEMIPFGAGRRICAGMSLGLRMVTFMTATLVHGFDWKLPNGVVAEKLDMEEAYGLTLQRAVPLMVLPVPRLAKQAYGKHEKL

SEQ ID NO: 14 - protein, 97 aa, Sorghum bicolor
DRFLPGGSHAGVDVKGSDFELIPFGAGRRICAGLSWGLRMVTLMTATLVHALDWDLADGMTAYKLDMEEAYGLTLQRAVPLMVRPAPRLLPSAYAAE

SEQ ID NO: 15 - protein, 97 aa, Zea mays
DRFLPGGSHAGVDVKGSEFELIPFGAGRRICAGLSWGLRMVSLMTATLVHALDWDLADGMTADKLDMEEACGLTLQRAVPLKVRPAPRLLPSAYAAE

SEQ ID NO: 16 - protein, 98 aa, Allium cepa
ERFLGGGGYETVDLKGNDFELIPFGAGRRVCAGLSLGLRMVQFLTATLVHGFDWKLVDGQSAEKLDMEEAYGLPLQRAVPLMVRPVPRLDEKAYHVVV

SEQ ID NO: 17 - protein, 96 aa, Antirrhinum majus
ERFLKGGEKPNVDVRGNDFELIPFGAGRRICAGMSLGIRMVQLLTATLIHAFDFDLADGQLPESLNMEEAYGLTLQRADPLVVHPKPRLAPHVYQT

SEQ ID NO: 18 - protein, 94 aa, Torenia hybrid
ERFLTGGEKADVDVKGNDFELIPFGAGRRICAGVGLGIRMVQLLTASLIHAFDLDLANGLLPQNLNMEEAYGLTLQRAEPLLVHPRLRLATHVY

SEQ ID NO: 19 - protein, 99 aa, Malus domestica
ERFMSGGEKPNVDIRGNDFEVIPFGAGRRICAGMSLGLRMVSLMTATLVHGFDWTLADGLTPEKLNMDEAYGLTLQRAAPLMVHPRNRLAPHAYNASSS

SEQ ID NO: 20 - protein, 99 aa, Matthiola incana
ERFLPGGEKFGVDVKGSDFELIPFGAGRRICAGLSLGLRTIQLLTATLVHGFEWELAGGVTPEKLNMEETYGITVQRAVPLIVHPKPRLALNVYGVGSG

SEQ ID NO: 21 - protein, 94 aa, Pelargonium hortorum
ERFLPGSEKENVDVKGNDFELIPFGAGRRICAGMSLGLRMVQLLTATLLHAFNWDLPQGQIPQELNMDEAYGLTLQRASPLHVRPRPRLPSHLY